SEQUENCE LISTING (9 sequences)

SEQ ID NO: 1 - protein, 380 aa, Homo sapiens
  misc_feature: Human IL-13 Receptor
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 2 - protein, 425 aa, Homo sapiens
  misc_feature: Human Orexin Receptor 1
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPLLFKSTARRARGSILGIWAVSLAIMVPQAAVMECSSVLPELANRTRLFSVCDERWADDLYPKIYHSCFFIVTYLAPLGLMAMAYFQIFRKLWGRQIPGTTSALVRNWKRPSDQLGDLEQGLSGEPQPRGRAFLAEVKQMRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSLQSRCSISKISEHVVLTSVTTVLP

SEQ ID NO: 3 - protein, 444 aa, Homo sapiens
  misc_feature: Human Orexin Receptor 2
MSGTKLEDSPPCRNWSSASELNETQEPFLNPTDYDDEEFLRYLWREYLHPKEYEWVLIAGYIIVFVVALIGNVLVCVAVWKNHHMRTVTNYFIVNLSLADVLVTITCLPATLVVDITETWFFGQSLCKVIPYLQTVSVSVSVLTLSCIALDRWYAICHPLMFKSTAKRARNSIVIIWIVSCIIMIPQAIVMECSTVFPGLANKTTLFTVCDERWGGEIYPKMYHICFFLVTYMAPLCLMVLAYLQIFRKLWCRQIPGTSSVVQRKWKPLQPVSQPRGPGQPTKSRMSAVAAEIKQIRARRKTARMLMVVLLVFAICYLPISILNVLKRVFGMFAHTEDRETVYAWFTFSHWLVYANSAANPIIYNFLSGKFREEFKAAFSCCCLGVHHRQEDRLTRGRTSTESRKSLTTQISNFDNISKLSEQVVLTSISTLPAANGAGPLQNW

SEQ ID NO: 4 - protein, 422 aa, Homo sapiens
  misc_feature: Melanin-concentrating Hormone Receptor 1
MSVGAMKKGVGRAVGLGGGSGCQATEEDPLPDCGACAPGQGGRRWRLPQPAWVEGSSARLWEQATGTGWMDLEASLLPTGPNASNTSDGPDNLTSAGSPPRTGSISYINIIMPSVFGTICLLGIIGNSTVIFAVVKKSKLHWCNNVPDIFIINLSVVDLLFLLGMPFMIHQLMGNGVWHFGETMCTLITAMDANSQFTSTYILTAMAIDRYLATVHPISSTKFRKPSVATLVICLLWALSFISITPVWLYARLIPFPGGAVGCGIRLPNPDTDLYWFTLYQFFLAFALPFVVITAAYVRILQRMTSSVAPASQRSIRLRTKRVTRTAIAICLVFFVCWAPYYVLQLTQLSISRPTLTFVYLYNAAISLGYANSCLNPFVYIVLCETFRKRLVLSVKPAAQGQLRAVSNAQTADEERTESKGT

SEQ ID NO: 5 - protein, 340 aa, Homo sapiens
  misc_feature: Melanin-concentrating Hormone Receptor 2
MNPFHASCWNTSAELLNKSWNKEFAYQTASVVDTVILPSMIGIICSTGLVGNILIVFTIIRSRKKTVPDIYICNLAVADLVHIVGMPFLIHQWARGGEWVFGGPLCTIITSLDTCNQFACSAIMTVMSVDRYFALVQPFRLTRWRTRYKTIRINLGLWAASFILALPVWVYSKVIKFKDGVESCAFDLTSPDDVLWYTLYLTITTFFFPLPLILVCYILILCYTWEMYQQNKDARCCNPSVPKQXVMKLTKMVLVLVVVFILSAAPYHVIQLVNLQMEQPTLAFYVGYYLSICLSYASSSINPFLYILLSGNFQKRLPQIQRRATEKEINNMGNTLKSHF

SEQ ID NO: 6 - protein, 802 aa, Homo sapiens
  misc_feature: Fibroblast Growth Factor Receptor - 4
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 7 - protein, 806 aa, Homo sapiens
  misc_feature: Fibroblast Growth Factor Receptor - 3
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 8 - protein, 821 aa, Homo sapiens
  misc_feature: Fibroblast Growth Factor Receptor - 2
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 9 - protein, 759 aa, Homo sapiens
  misc_feature: Fibroblast Growth Factor Receptor - 1
MSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVAL

(19) United States
(12) Patent Application Publication
(10) Pub. No.: US 2005/0060766 A1
(43) Pub. Date: Mar. 17, 2005

(54) Expression of dominant negative transmembrane receptors in the milk of transgenic animals

(76) Inventor: Li-How Chen, Acton, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/941,486

(22) Filed: Sep. 15, 2004

Related U.S. Application Data
(60) Provisional application No. 60/503,153, filed on Sep. 15, 2003.

(51) Int. Cl. A01K 67/027; C12N 15/85

(52) U.S. Cl. 800/21; 435/455

(57) ABSTRACT

The present invention provides data to demonstrates the transgenic mammal production of membrane spanning receptor proteins in the milk of transgenic animals, offering a method of production of these proteins and dominant negative versions thereof for use as therapeutic molecules.

### FIELD OF THE INVENTION

The present invention relates to improved methods for the production of transgenic animals capable of expressing desired transmembrane receptor constructs in the milk of transgenic mammals. More specifically, the current invention provides a method to improve production of animals transgenic for the expression of transmembrane receptor proteins and/or dominant negative transmembrane receptor proteins useful as therapeutic molecules.

### BACKGROUND OF THE INVENTION

The present invention relates generally to the field of nuclear transfer and the creation of desirable transgenic animals. More particularly, it concerns methods for generating transmembrane receptor proteins in transgenic animals.

The development of technology capable of generating transgenic animals provides a means for exceptional precision in the production of animals that are engineered to carry specific traits or are designed to express certain proteins or other molecular compounds. That is, transgenic animals are animals that carry a gene that has been deliberately introduced into somatic and/or germline cells at an early stage of development. As the animals develop and grow the protein product or specific developmental change engineered into the animal becomes apparent.

The ability to discover lead chemical matter for novel therapeutic targets is the first critical step in drug discovery programs for most pharmaceutical companies. Recent advances in cell biology, genomic sequencing, and transgenics have allowed dissection of signal transduction pathways, as well as novel biochemical control points, facilitating identification of potential novel opportunities for small molecule drug intervention at a rate unprecedented in the industry.

Along this line G-protein-coupled receptors (GPCRs) are a major class of target for the pharmaceutical industry. GPCRs are a superfamily of 7-transmembrane receptor proteins that have critical functions in numerous autocrine, paracrine, and endocrine signaling systems. These proteins transduce the binding of extracellular ligands and hormones into intracellular signaling events through modulation of guanine nucleotide binding regulatory proteins (G-proteins). Traditional drug discovery programs targeting GPCRs have relied on the use of whole animals or tissue preparations from native sources as a starting point to perform screens of synthetic/medicinal or natural product libraries in biological or pharmacological assays. Due to expression problems associated with the very nature of transmembrane proteins, transmembrane receptor proteins have been exceptionally hard to express or purify in useable amounts. (Loisel et al., 1997).

Those working in the field have been unsuccessful in producing any appreciable amounts of soluble transmembrane receptor or dominant negative versions thereof as stand alone therapeutic molecules. For example, much effort has been expended on discovering a surrogate small molecule ligand for the 166-residue hematopoietic growth hormone erythropoietin (EPO) and its cytokine receptor. A 20-residue cyclic peptide unrelated in sequence to the natural EPO ligand has been identified and studied extensively (Livnah et al., 1996), but this reduced-size peptide has not translated into a drug itself, nor has it helped make a receptor protein available for the development of a therapeutic molecule.

Prior to the present invention the techniques available for the generation of transgenic domestic animals capable of producing transmembrane receptor proteins were inefficient and/or were not able to produce the desired recombinant protein in anything nearing a commercially viable scale. During the development of a transgenic founder line carrying a receptor transmembrane DNA sequences of interest there are a variety of problems. Typically, the transgene may either be not incorporated at all, or incorporated but not expressed. A further problem is the possibility of inaccurate regulation due to positional effects. This refers to the variability in the level of gene expression and the accuracy of gene regulation between different founder animals produced with the same transgenic constructs. Thus, it is not uncommon to generate a large number of founder animals and often confirm that less than 5% express the transgene in a manner that warrants the maintenance of the transgenic line.

Additionally, the efficiency of generating transgenic domestic animals is low, with efficiencies of 1 in 100 offspring generated being transgenic not uncommon (Wall et al., 1997). As a result the cost associated with generation of transgenic animals can be as much as 250-500 thousand dollars per expressing animal (Wall et al., 1997).

Prior art methods have typically used embryonic cell types in cloning procedures. This includes work by Campbell et al (NATURE 1996) and Stice et al (BIOL. REPROD. 1996). In both of those studies, embryonic cell lines were derived from embryos of less than 10 days of gestation. In both studies, the cells were maintained on a feeder layer to prevent overt differentiation of the donor cell to be used in the cloning procedure. The present invention uses differentiated cells. It is considered that embryonic cell types could also be used in the methods of the current invention along with cloned embryos starting with differentiated donor nuclei.

Thus although transgenic animals have been produced by various methods in several different species, methods to readily and reproducibly produce transgenic animals capable of expressing a desired transmembrane protein in high quantity or demonstrating the genetic change caused by the insertion of the transgene(s) at reasonable costs are still lacking. Previous attempts at expressing include engineering membrane associated proteins with the transmembrane domains deleted, thus leaving the extracellular portions which can bind to ligands. (St. Croix et al., United States Patent Application 20030017157). Such soluble forms of transmembrane receptor proteins can be used to compete with natural forms for binding to ligand. It is possible that such soluble fragments can act as inhibitors, but it is uncertain if they will truly offer the capability to truly compete with native transmembrane receptors retaining their transmembrane sequence.

With regard to asthma and associated respiratory ailments epidemiological studies clearly demonstrate that the prevalence of allergic diseases has increased, and that the higher diagnosis rates are due not simply to changes in diagnostic fashion or improvements in detection. Additionally, the increasing recognition that allergic rhinitis and allergic asthma frequently co-exist has led to the concept that these seemingly separate disorders are manifestations of the same disease expressed in either the upper or the lower airways.

Many treatments for asthma today do not target the mechanisms that underlie the progression of the disease itself, and, in some cases, are associated with significant side-effects and decreased efficacy after prolonged use. Despite the therapeutic advances made over the past 25 years, the prevalence and severity of asthma has risen substantially and there is clearly a need to develop new drugs against novel therapeutic targets. The commercial potential for a new and effective asthma medication is very significant with the current market size for asthma drugs estimated to be in excess of US $5 billion.

While a range of new therapies that target various aspects of asthma pathology are currently in clinical development, a significant body of data points to the interaction of IL-13 with its receptor as the key interaction, occurring upstream of other cytokine and non-cytokine based targets. However, production of a dysfunctional transmembrane receptor to IL-13, as a potential therapeutic pathway for the treatment of asthma has not been pursued or suggested.

Accordingly, a need exists for improved methods for the recombinant expression of transmembrane receptor proteins will allow an increase in production efficiencies in the development of transgenic animals, particularly with regard to the production of a molecule that may offer an additional therapeutic option for the treatment of asthma or related allergy conditions.

### SUMMARY OF THE INVENTION

Briefly stated, the current invention provides a method for expressing transmembrane proteins in a transgenic recombinant system. The method of the invention involves cloning a non-human mammal transgenic for a desired receptor transmembrane receptor protein through a nuclear transfer process comprising: obtaining desired differentiated mammalian cells to be used as a source of donor nuclei; obtaining at least one oocyte from a mammal of the same species as the cells which are the source of donor nuclei; enucleating the at least one oocyte; transferring the desired differentiated cell or cell nucleus into the enucleated oocyte; simultaneously fusing and activating the cell couplet to form a transgenic embryo; culturing the activated transgenic embryo(es) until greater than the 2-cell developmental stage; and finally transferring the transgenic embryo into a suitable host mammal such that the embryo develops into a fetus. Typically, the above method is completed through the use of a donor cell nuclei in which a desired gene, encoding a transmembrane receptor protein of interest has been inserted, removed or modified prior to insertion of said differentiated mammalian cell or cell nucleus into said enucleated oocyte. Also of note is the fact that the oocytes used are preferably matured in vitro prior to enucleation.

In addition, the current invention provides for the transgenic production of transmembrane receptors including: the IL-13 receptor, the Fibroblast Growth Factor Receptors 1 through 4, the CFTR receptor, the orexin receptor, the melanin concentrating hormone receptor, the CD-4 receptor, as well as dominant negative versions of all of the above. The current invention demonstrates that many different transmembrane proteins could be produced in the transgenic milk. This capability is unique to the recombinant mammal transgenic expression system. The current invention also provides for the expression and manufacture of a dominant negative transmembrane proteins capable of inhibiting receptor function. This expression allows the use of the expressed molecules to form the basis of a new therapeutic approach targeting of disease pathologies by intervening in signal transduction pathways dependent upon transmembrane receptors.

According to a preferred embodiment the dominant negative transmembrane receptor protein is made so through the elimination of the functionality of one or more tyrosine kinase sites in the protein of interest. Other sites that can be altered to eliminate physiological function include active serine kinase sites important in the function of a transmembrane receptor protein of interest.

Moreover, the method of the current invention also provides for optimizing the generation of transgenic animals through the use of caprine oocytes, arrested at the Metaphase-II stage, that were enucleated and fused with donor somatic cells and simultaneously activated. Analysis of the milk of one of the transgenic cloned animals showed high-level production of human of the desired target transgenic protein product.

It is also important to point out that cells, tissues, and organs can be isolated from cloned offspring as well. This process can provide a source of “materials” for many medical and veterinary therapies including cell and gene therapy. If the cells are transferred back into the animal in which the cells were derived, then immunological rejection is averted. Also, because many cell types can be isolated from these clones, other methodologies such as hematopoietic chimericism can be used to avoid immunological rejection among animals of the same species as well as between species.

### BRIEF DESCRIPTION OF THE DRAWINGS

FIG. 1 Shows A Generalized Diagram of the Process of Creating Cloned Animals through Nuclear Transfer.

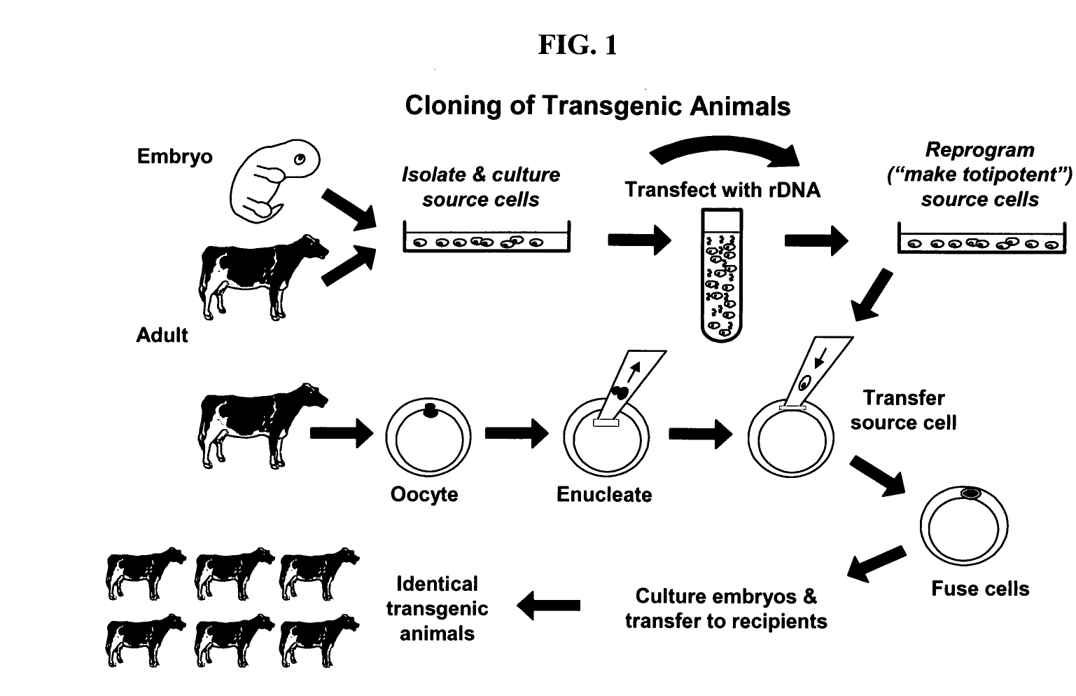

FIG. 2 Shows the construction of the IL-13 receptor transgene.

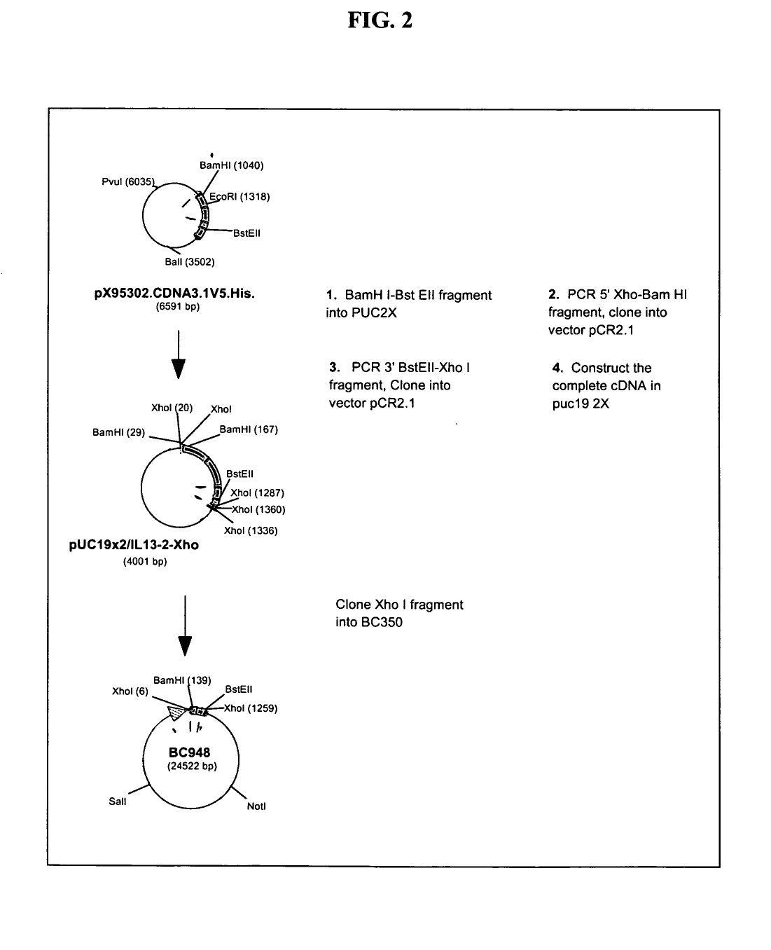

FIG. 3 Shows the expression of IL13 receptor in the milk of transgenic mice. Lanes 1-8, total milk from eight founder mice BC894-4, BC894-79, BC894-81, BC894-96, BC894-104, BC894-114A, BC894-114B and BC894-116, respectively. Lanes 9 and 10, the lipid fraction of mice 1 and 2, respectively. M, molecular weight maker. N, negative milk.

### DESCRIPTION OF THE PREFERRED EMBODIMENT

The following abbreviations have designated meanings in the specification:

Abbreviation Key:

| | |
|---|---|
| Somatic Cell Nuclear Transfer | (SCNT) |
| Cultured Inner Cell Mass Cells | (CICM) |
| Nuclear Transfer | (NT) |
| Synthetic Oviductal Fluid | (SOF) |
| Fetal Bovine Serum | (FBS) |
| Polymerase Chain Reaction | (PCR) |
| Bovine Serum Albumin | (BSA) |

Explanation of Terms:

Caprine—Of or relating to various species of goats.

Reconstructed Embryo—A reconstructed embryo is an oocyte that has had its genetic material removed through an enucleation procedure. It has been “reconstructed” through the placement of genetic material of an adult or fetal somatic cell into the oocyte following a fusion event.

Fusion Slide—A glass slide for parallel electrodes that are placed a fixed distance apart. Cell couplets are placed between the electrodes to receive an electrical current for fusion and activation.

Cell Couplet—An enucleated oocyte and a somatic or fetal karyoplast prior to fusion and/or activation.

Cytocholasin-B—A metabolic product of certain fungi that selectively and reversibly blocks cytokinesis while not effecting karyokinesis.

Cytoplast—The cytoplasmic substance of eukaryotic cells.

Dominant Negative Effect—The mutant receptor or altered amino acid sequence can dimerize with the wildtype receptor/ligand, but intracellular signaling cannot be activated because of the absence or alteration in a key domain region (ex: a tyrosine kinase domain is missing from the mutant receptor). Therefore, the cells with this mutation will be unable to respond in the presence of ligand.

Karyoplast—A cell nucleus, obtained from the cell by enucleation, surrounded by a narrow rim of cytoplasm and a plasma membrane.

Somatic Cell—Any cell of the body of an organism except the germ cells.

Parthenogenic—The development of an embryo from an oocyte without the penetrance of sperm

Transgenic Organism—An organism into which genetic material from another organism has been experimentally transferred, so that the host acquires the genetic traits of the transferred genes in its chromosomal composition.

Somatic Cell Nuclear Transfer—Also called therapeutic cloning, is the process by which a somatic cell is fused with an enucleated oocyte. The nucleus of the somatic cell provides the genetic information, while the oocyte provides the nutrients and other energy-producing materials that are necessary for development of an embryo. Once fusion has occurred, the cell is totipotent, and eventually develops into a blastocyst, at which point the inner cell mass is isolated.

Significant advances in nuclear transfer have occurred since the initial report of success in the sheep utilizing somatic cells (Wilmut et al., 1997). Many other species have since been cloned from somatic cells (Baguisi et al., 1999 and Cibelli et al., 1998) with varying degrees of success. Numerous other fetal and adult somatic tissue types (Zou et al., 2001 and Wells et al., 1999), as well as embryonic (Yang et al., 1992; Bondioli et al., 1990; and Meng et al., 1997), have also been reported. The stage of cell cycle that the karyoplast is in at time of reconstruction has also been documented as critical in different laboratories methodologies (Kasinathan et al., Biol. Reprod. 2001; Lai et al., 2001; Yong et al., 1998; and Kasinathan et al., Nature Biotech. 2001). However, there is quite a large degree of variability in the sequence, timing and methodology used for fusion and activation.

Prior art techniques rely on the use of blastomeres of early embryos for nuclear transfer procedure. This approach is limited by the small numbers of available embryonic blastomeres and by the inability to introduce foreign genetic material into such cells. In contrast, the discoveries that differentiated embryonic, fetal, or adult somatic cells can function as karyoplast donors for nuclear transfer have provided a wide range of possibilities for germline modification. According to the current invention, the use of recombinant somatic cell lines for nuclear transfer, and improving this procedures efficiency by increasing the number of available cells through the use of “reconstructed” embryos, not only allows the introduction of transgenes by traditional transfection methods into more transgenic animals but also increases the efficiency of transgenic animal production substantially while overcoming the problem of founder mosaicism.

We have previously shown that simultaneous electrical fusion and activation can successfully produce live offspring in the caprine species, and other animals. Donor karyoplasts were obtained from a primary fetal somatic cell line derived from a 40-day transgenic female fetus produced by artificial insemination of a negative adult female with semen from a transgenic male. Live offspring were produced with two nuclear transfer procedures. In one protocol, caprine oocytes at the arrested Metaphase-II stage were enucleated, electrofused with donor somatic cells and simultaneously activated. In the second protocol, activated in vivo caprine oocytes were enucleated at the Telophase-II stage, electrofused with donor karyoplasts and simultaneously activated a second time to induce genome reactivation. Three healthy identical female offspring were born. Genotypic analyses confirmed that all cloned offspring were derived from the donor cell line. Analysis of the milk of one of the transgenic cloned animals showed high-level production of human transmembrane receptor proteins. Thus, through the methodology and system employed in the current invention transgenic animals, goats, were generated by somatic cell nuclear transfer and were shown to be capable of producing a target therapeutic receptor protein in the milk of a cloned animal.

Although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

GPCRs

Typically, GPCRs have been classified and receptor subtypes identified via the observation of pharmacological differences in the affinities of agonists and antagonists in radiolabel binding assays. With the advent of modern genomics, screening of recombinant human receptors of known subtype expressed in specific cell lines has become the norm for lead discovery programs.

A typical discovery scenario of the current art might include the use of a radioligand membrane displacement assay, followed by a cellular reporter secondary assay. Regardless of the assay employed a series of single cell clones expressing high levels of the receptor of interest must be identified and made available for molecular screening, and this is often most easily accomplished using a reporter gene readout (Stables et al., 1999). The alternative approach involves picking clones via whole cell radio-ligand binding assays. The latter approach is free of patent restrictions, but is more labor intensive. The process usually begins with transfection of the cDNA for the receptor of interest into a stable cell line co-expressing a reporter gene under the control of a promoter that is modulated by the receptor-dependent signal transduction pathway. Activation of the receptor of interest by its ligand or an agonist ultimately results in the transcription of the reporter gene whose activity is easily measured. This activity is used to identify a receptor-expressing, stable, clonal cell line, as usually the amplitude of the reporter signal correlates with receptor expression levels. Once a positive clone is identified, it is expanded, and the assay format is chosen. Displacement assays are of two general types: filtration-based radio-ligand binding and SPA. The detection of active compounds by displacement presents a simple well-defined system, and therefore allows for detailed affinity and structure-activity relationship (SAR) studies to be performed (Rosati et al. 1998). However, according to the prior art, it has not been possible to prepare and express the transmembrane receptor itself, or a dominant negative version of it for use as a potentially therapeutic molecule.

Because of the historically low success rate, targeting protein—receptor interactions is an area the biotechnology industry largely avoids. An example of a protein/protein interaction is a cytokine or growth factor engaging its receptor target. Biologically, these play important roles controlling key events in signal transduction, cell trafficking, and adhesion, and are therefore potentially attractive as points of intervention in autoimmune diseases, cancer, asthma, allergy, and others.

Expression of Transmembrane Receptor Proteins

One unique physiological feature of the lactating mammary epithelial cells is that they secrete lipids into the milk. The lipids are secreted epically as milk fat globules, fat droplets enveloped by a membrane of phospholipids and the proteins. A number of cellular membrane proteins are found in the membrane fraction of the milk fat globules. We provide in the current invention a method that utilizes this secretory pathway as a tool for the production of recombinant transmembrane proteins from the milk of transgenic animals. When a protein with one or more transmembrane domains is expressed from a transgene in the mammary gland, the mammary epithelial cells may be able to “secrete” it in the milk fat globules thus the recombinant protein may be harvested from the milk. This will make the transgenic milk production the only system that is able to secrete transmembrane proteins and afford the practitioners of the current invention the opportunity to potentially produce many classes of transmembrane proteins such as the channels proteins, the cell surface receptors, the drug resistance regulators that other protein expression systems fail to offer. The current invention provides for the expression of trans-membrane proteins such as the IL-13 receptor, and a dominant negative version thereof in the milk of transgenic animals.

A Transgenic Dominant Negative IL-13 Receptor for the Treatment of Asthma and Allergy

Current asthma management guidelines emphasize the importance of early intervention with inhaled corticosteroids as first-line anti-inflammatory therapy. Several studies have demonstrated that certain second generation of antihistamines possess anti-inflammatory activity. Studies were also conducted investigating their effects in combination with leukotriene receptor antagonists versus intranasal and/or inhaled corticosteroids in both allergic rhinitis and asthma. Amongst the novel anti-cytokine therapies, treatments with anti-IL-5, anti-IL-13, anti-TNF-α, as well as soluble IL-4 receptor antagonists are currently being studied in asthmatics.

Recent published studies in mice have highlighted the role of IL-13 in the development of allergic asthma. Mice primed to develop asthma-like symptoms showed reduction or ablation of such symptoms when treated with a truncated form of IL-13. Repeat administration of recombinant IL-13 to the airways of naive mice induced similar symptoms and confirmed the role of IL-13 in these pathologies.

These reports and a variety of other studies identify a central role for IL-13 in the development of mouse allergic airway disease and, by extension, human asthma. In humans recent collaborative studies have demonstrated IL-13 receptor expression in a variety of cells found in biopsies of human asthmatic lung. The data indicate that IL-13 plays an important role in the development of crucial features of airway disease. On this basis, the availability of a dominant negative IL-13 receptor available to compete with the ligands of the native IL-13 receptor or otherwise interfere with the components of the IL-13 signaling pathway, represents a novel therapeutic pathway for the therapeutic treatment of asthma or allergic rhinitis.

Construction of the IL-13 Receptor Transgene

IL-13 is a type 2 cytokine recently found to be necessary and sufficient to mediate allergic asthma in animal models. Neutralization of the IL-13 ligand with an IL-13 receptor was shown to completely block asthmatic phenotype which included the air way hypersensitivity, the IgE production and the mucus hypersecretion (SCIENCE, December 1998). According to the current invention we provide a dominant negative mutant of the IL-13 receptor that can be made by the transgenic expression system of the invention and thereafter delivered to the airway cells. Upon delivery the normal signal transduction path of IL-13 is blocked, leading to the inhibition of the receptor. The therapeutic outcome is the treatment of the asthma phenotype. We therefore chose to express IL-13 receptor as an example of producing membrane proteins in the milk as well as a the expression of a dominant negative membrane receptor in a way making it available for production as a therapeutic molecule.

To construct the transgene, the cDNA of the IL-13 receptor (obtained from Invitrogen) was subcloned into the cloning vector puc19-2X to introduce two Xho I sites, one 5′ to start codon and the other 3′ to the stop codon. The Xho I fragment of the IL-13 receptor cDNA was then cloned into BC350 to yield BC948. The BC948 transgene contained the entire IL-13 receptor conding region followed by a V5 tag and a HisC tag at its C-terminal. The Sal I/Not I fragment of BC948 was purified for microinjection. Transgenic founder mice were identified by PCR using IL-13 receptor transgene specific oligo pairs.

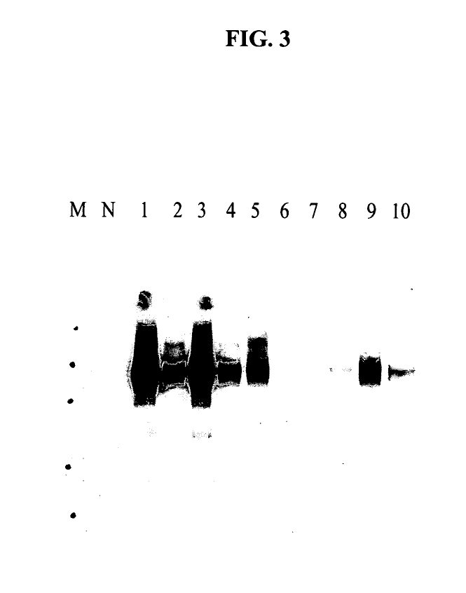

Expression of the IL-13 receptor in the milk was determined by western blotting using HRP conjugated anti-V5 tag antibodies. Of the seven female transgenic founder mice analyzed, 5 expressed IL-3 in their milk. The level of IL-13 receptor expression ranged from 0.1 to 0.25 mg/ml (FIG. 3).

The sequence of the human IL-13 receptor is known and was presented by several different authors in the field. Below is the amino sequence of human IL-13 Receptor:

Genbank/EMBL /DDBJ Accession No. NP_000631, from
the National Center for Biotechnology Information - human
IL-13 Receptor (380 amino acid residues); (Wu et al.,
(2003); and David et al., (2002))

| | | | | | | | |
|---|---|---|---|---|---|---|---|
| 1 | mafvclaigc | lytflisttf | gctsssdtei | kvnppqdfei | vdpgylgyly | lqwqpplsld | SEQ. ID. No. 1 |
| 61 | hfkectveye | lkyrnigset | wktiitknlh | ykdgfdlnkg | ieakihtllp | wqctngsevq | |
| 121 | sswaettywi | spqgipetkv | qdmdcvyynw | qyllcswkpg | igvlldtnyn | lfywyegldh | |
| 181 | alqcvdyika | dgqnigcrfp | yleasdykdf | yicvngssen | kpirssyftf | qlqnivkplp | |
| 241 | pvyltftres | sceiklkwsi | plgpiparcf | dyeieiredd | ttlvtatven | etytlkttne | |
| 301 | trqlcfvvrs | kvniycsddg | iwsewsdkqc | wegedlskkt | llrfwlpfgf | ililvifvtg | |
| 361 | lllrkpntyp | kmipeffcdt. | | | | | |

Cadherins

Cadherins constitute a family of cell surface transmembrane receptor proteins that are organized into eight groups. The best-known group of cadherins, called “classical cadherins,” plays a role in establishing and maintaining cell-cell adhesion complexes such as the adherens junctions. Classical cadherins function as clusters of dimers, and the strength of adhesion is regulated by varying both the number of dimers expressed on the cell surface and the degree of clustering. Classical cadherins bind to cytoplasmic adaptor proteins, called catenins, which link cadherins to the actin cytoskeleton. Cadherin clusters regulate intracellular signaling by forming a cytoskeletal scaffold that organizes signaling proteins and their substrates into a three-dimensional complex. Classical cadherins are essential for tissue morphogenesis, primarily by controlling specificity of cell-cell adhesion as well as changes in cell shape and movement. The cadherin superfamily consists of over 70 structurally related proteins, all of which share two properties: the extracellular regions of these proteins bind to calcium ions to fold properly (hence Ca, for calcium) and these proteins adhere to other proteins (hence, “adherin”). The cadherins are involved in cell-cell adhesion, cell migration, and signal transduction. The first group of cadherins discovered includes those found in the zonula adherens junctions formed between epithelial cells. These are now termed “classical cadherins” to distinguish them from their more distantly related family members. All classical cadherins are transmembrane receptors with a single membrane-spanning domain, five extracellular domains at the amino end of the protein, and a conserved cytoplasmic C-terminal tail.

In vertebrates, the five classical cadherins are termed E-, P-, N-, R-, and VE-cadherins, based on the sites where they were first discovered: epithelium, placenta, nerve, retina, and vascular endothelium, respectively. Classical cadherins function as clusters of dimers on the cell surface. These dimers bind to identical dimers on neighboring cells. The N- and R-cadherin pairs will also bind to each other (heterophilic binding). Cells can control their strength of adhesion by avidity modulation, which involves varying both the total number of receptors on the cell surface and the lateral diffusion of the receptors within the plasma membrane. Cadherins that are not clustered will not form strong adhesions with neighboring cells. There is direct evidence for the importance of cadherin clustering in cell-cell adhesion. The experiment that provided this evidence is based on the fact that the cadherin cytoplasmic tails are important for dimerization (Yap et al., 1997).

Classical cadherins play a significant role during development by controlling the strength of cell-cell adhesion and by providing a mechanism for specific cell-cell recognition. For example, during development, E-cadherins are expressed when the lastocyst forms, and are thought to increase cell-cell adhesion when tight junctions form and epithelial cells subsequently polarize in the developing embryo. Not surprisingly, genetic knockout of E-cadherin genes is lethal early in development (Larue et al., 1994). Functional mutations or knockout of other cadherin family members affect development of a wide variety of organs including brain, spinal chord, lung, and kidney. An important theme common to all of these developmental events is a process of cellular movement known as invagination. For example, the first nervous tissue arises in vertebrates when the cells comprising the ectoderm form a ridge along the outer surface of the embryo that deepens into a cleft and then pinches off to form the neural tube. To form this tube, epithelial cells must constrict their apical domains and bend inward, forming a groove, then dissociate and move to new locations to close the tube. Similar movements occur in the formation of many ectodermally derived tissues, and all require variations in the types of cell-cell contacts. Deletion of cadherin genes results in a wide variety of developmental abnormalities, such as poor motor skills due to mistargeted neurons, which also result from errors in epithelial invaginations. (Fesenko, 2001).

Other Molecules of Interest

Orexin Receptors

Genbank/EMBL /DDBJ Accession No. NP_001516, from the National
Center for Biotechnology Information - human orexin receptor 1,
(Sakurai, T., et at., (1998)) (425 amino acids).

| | | | | | | | |
|---|---|---|---|---|---|---|---|
| 1 | mepsatpgaq | mgvppgsrep | spvppdyede | flrylwrdyl | ypkqyewvli | aayvavfvva | SEQ. ID.: 2 |
| 61 | lvgntlvcla | vwrnhhmrtv | tnyfivnlsl | advlvtaicl | pasllvdite | swlfghalck | |
| 121 | vipylqavsv | svavltlsfi | aldrwyaich | pllfkstarr | argsilgiwa | vslaimvpqa | |
| 181 | avmecssvlp | elanrtrlfs | vcderwaddl | ypkiyhscff | ivtylaplgl | mamayfqifr | |
| 241 | klwgrqipgt | tsalvrnwkr | psdqlgdleq | glsgepqprg | raflaevkqm | rarrktakml | |
| 301 | mvvllvfalc | ylpisvlnvl | krvfgmfrqa | sdreavyacf | tfshwlvyan | saanpiiynf | |
| 361 | lsgkfreqfk | aafscclpgl | gpcgslkaps | prssashksl | slqsrcsisk | isehvvltsv | |
| 421 | ttvlp | | | | | | |

Genbank/EMBL /DDBJ Accession No. NP_001517, from the National
Center for Biotechnology Information - human orexin receptor 2, (de
Lecea, L., et al., (1998)) (444 amino acids).

| | | | | | | | |
|---|---|---|---|---|---|---|---|
| 1 | msgtkledsp | pcrnwssase | lnetqepfln | ptdyddeefl | rylwreylhp | keyewvliag | SEQ. ID.: 3 |
| 61 | yiivfvvali | gnvlvcvavw | knhhmrtvtn | yfivnlslad | vlvtitclpa | tlvvditetw | |
| 121 | ffgqslckvi | pyiqtvsvsv | svltlscial | drwyaichpl | mfkstakrar | nsiviiwivs | |
| 181 | ciimipqaiv | mecstvfpgl | ankttlftvc | derwggeiyp | kmyhicfflv | tymaplclmv | |
| 241 | laylqifrkl | wcrqipgtss | vvqrkwkplq | pvsqprgpgq | ptksrmsava | aeikqirarr | |
| 301 | ktarmlmvvl | lvfaicylpi | silnvlkrvf | gmfahtedre | tvyawftfsh | wlvyansaan | |
| 361 | piiynflsgk | freefkaafs | ccclgvhhrq | edrltrgrts | tesrkslttq | isnfdniskl | |
| 421 | seqvvltsis | tlpaangagp | lqnw | | | | |

Melanin Concentrating Hormone Receptors

Genbank/EMBL /DDBJ Accession No. NP_005288, from the National
Center for Biotechnology Information - Melanin-concentrating hormone
receptor 1 (Pissios, P., et al., (2003)) (422 amino acids).

| | | | | | | | |
|---|---|---|---|---|---|---|---|
| 1 | msvgamkkgv | gravglgggs | gcqateedpl | pdcgacapgq | ggrrwrlpqp | awvegssarl | SEQ. ID.: 4 |
| 61 | weqatgtgwm | dleasllptg | pnasntsdgp | dnltsagspp | rtgsisyini | impsvfgtic | |
| 121 | llgiignstv | ifavvkkskl | hwcnnvpdif | iinlsvvdll | fllgmpfmih | qlmgngvwhf | |
| 181 | getmctlita | mdansqftst | yiltamaidr | ylatvhpiss | tkfrkpsvat | lvicllwals | |
| 241 | fisitpvwly | arlipfpgga | vgcgirlpnp | dtdlywftly | qfflafalpf | vvitaayvri | |
| 301 | lqrmtssvap | asqrsirlrt | krvtrtaiai | clvffvcwap | yyvlqltqls | isrptltfvy | |
| 361 | lynaaislgy | ansclnpfvy | ivlcetfrkr | lvlsvkpaaq | gqlravsnaq | tadeertesk | |
| 421 | gt | | | | | | |

Genbank/EMBL /DDBJ Accession No. NP_115892, from the National
Center for Biotechnology Information - Melanin-concentrating hormone
receptor 2 (Hill J., et al., (2001)) (340 amino acids).

| | | | | | | | |
|---|---|---|---|---|---|---|---|
| 1 | mnpfhascwn | tsaellnksw | nkefayqtas | vvdtvilpsm | igiicstglv | gnilivftii | SEQ. ID.: 5 |
| 61 | rsrkktvpdi | yicnlavadl | vhivgmpfli | hqwarggewv | fggplctiit | sldtcnqfac | |
| 121 | saimtvmsvd | ryfalvqpfr | ltrwrtrykt | irinlglwaa | sfilalpvwv | yskvikfkdg | |
| 181 | vescafdlts | pddvlwytly | ltittfffpl | plilvcyili | lcytwemyqq | nkdarccnps | |
| 241 | vpkqxvmklt | kmvlvlvvvf | ilsaapyhvi | qlvnlqmeqp | tlafyvgyyl | siclsyasss | |
| 301 | inpflyills | gnfqkrlpqi | qrratekein | nmgntlkshf | | | |

Fibroblast Growth Factor Receptor—Family

Genbank/EMBL /DDBJ Accession No. P22455, from the National Center
for Biotechnology Information - Fibroblast Growth Factor Receptor - 4
(Partanen J., et al., (1991)) (802 amino acids).

| | | | | | | | |
|---|---|---|---|---|---|---|---|
| 1 | mrlllallgv | llsvpgppvl | sleaseevel | epclapsleq | qeqeltvalg | qpvrlccgra | SEQ. ID.: 6 |
| 61 | ergghwykeg | srlapagrvr | gwrgrleias | flpedagryl | clargsmivl | qnltlitgds | |
| 121 | ltssnddedp | kshrdpsnrh | sypqqapywt | hpqrmekklh | avpagntvkf | rcpaagnptp | |
| 181 | tirwlkdgqa | fhgenriggi | rlrhqhwslv | mesvvpsdrg | tytclvenav | gsirynylld | |
| 241 | vlersphrpi | lqaglpantt | avvgsdvell | ckvysdaqph | iqwlkhivin | gssfgadgfp | |
| 301 | yvqvlktadi | nssevevlyl | rnvsaedage | ytclagnsig | lsyqsawltv | lpeedptwta | |
| 361 | aapearytdi | ilyasgslal | avilliagly | rgqalhgrhp | rppatvqkls | rfplarqfsl | |
| 421 | esgssgksss | slvrgvrlss | sgpallaglv | sldlpldplw | efprdrlvlg | kplgegcfgq | |
| 481 | vvraeafgmd | parpdqastv | avkmlkdnas | dkdladlvse | mevmkligrh | kniinllgvc | |
| 541 | tqegplyviv | ecaakgnlre | flrarrppgp | dlspdgprss | egplsfpvlv | scayqvargm | |
| 601 | qylesrkcih | rdlaarnvlv | tednvmkiad | fglargvhhi | dyykktsngr | lpvkwmapea | |
| 661 | lfdrvythqs | dvwsfgillw | eiftlggspy | pgipveelfs | llreghrmdr | pphcppelyg | |
| 721 | lmrecwhaap | sqrptfkqlv | ealdkvllav | seeyldlrlt | fgpyspsggd | asstcsssds | |
| 781 | vfshdplplg | sssfpfgsgv | qt | | | | |

Genbank/EMBL /DDBJ Accession No. P22607, from the National Center
for Biotechnology Information - Fibroblast Growth Factor Receptor - 3
(Murgue, B., et al., (1991)) (806 amino acids).

| | | | | | | | |
|---|---|---|---|---|---|---|---|
| 1 | mgapacalal | cvavaivaga | sseslgteqr | vvgraaevpg | pepgqqeqlv | fgsgdavels | SEQ. ID.: 7 |
| 61 | cpppgggpmg | ptvwvkdgtg | lvpservlvg | pqrlqvlnas | hedsgayscr | qrltqrvlch | |
| 121 | fsvrvtdaps | sgddedgede | aedtgvdtga | pywtrpermd | kkllavpaan | tvrfrcpaag | |
| 181 | nptpsiswlk | ngrefrgehr | iggiklrhqq | wslvmesvvp | sdrgnytcvv | enkfgsirqt | |
| 241 | ytldvlersp | hrpilqaglp | anqtavlgsd | vefhckvysd | aqphiqwlkh | vevngskvgp | |
| 301 | dgtpyvtvlk | taganttdke | levlslhnvt | fedageytcl | agnsigfshh | sawlvvlpae | |
| 361 | eelveadeag | svyagilsyg | vgfflfilvv | aavtlcrlrs | ppkkglgspt | vhkisrfplk | |
| 421 | rqvslesnas | mssntplvri | arlssgegpt | lanvselelp | adpkwelsra | rltlgkplge | |
| 481 | gcfgqvvmae | aigidkdraa | kpvtvavkml | kddatdkdls | dlvsememmk | migkhkniin | |
| 541 | llgactqggp | lyvlveyaak | gnlreflrar | rppgldysfd | tckppeeqlt | fkdlvscayq | |
| 601 | vargmeylas | qkcihrdlaa | rnvlvtednv | mkiadfglar | dvhnldyykk | ttngrlpvkw | |
| 661 | mapealfdrv | ythqsdvwsf | gvllweiftl | ggspypgipv | eelfkllkeg | hrmdkpanct | |
| 721 | hdlymimrec | whaapsqrpt | fkqlvedldr | vltvtstdey | ldlsapfeqy | spggqdtpss | |
| 781 | sssgddsvfa | hdllppapps | sggsrt | | | | |

Genbank/EMBL /DDBJ Accession No. P21802, from the National Center
for Biotechnology Information - Fibroblast Growth Factor Receptor - 2
(Dionne C. A., et al., (1990)) (821 amino acids).

| | | | | | | | |
|---|---|---|---|---|---|---|---|
| 1 | mvswgrficl | vvvtmatlsl | arpsfslved | ttlepeeppt | kyqisqpevy | vaapgeslev | SEQ. ID.: 8 |
| 61 | rcllkdaavi | swtkdgvhlg | prmrtvlige | ylqikgatpr | dsglyactas | rtvdsetwyf | |
| 121 | mvnvtdaiss | gddeddtdga | edfvsensnn | krapywtnte | kmekrlhavp | aantvkfrcp | |
| 181 | aggnpmptmr | wlkngkefkq | ehriggykvr | nqhwslimes | vvpsdkgnyt | cvveneygsi | |
| 241 | nhtyhldvve | rsphrpilqa | glpanastvv | ggdvefvckv | ysdaqphiqw | ikhvekngsk | |
| 301 | ygpdglpylk | vlkaagvntt | dkeievlyir | nvtfedagey | tclagnsigi | sfhsawltvl | |
| 361 | papgrekeit | aspdyleiai | ycigvfliac | mvvtvilcrm | knttkkpdfs | sqpavhkltk | |
| 421 | riplrrqvtv | saessssmns | ntplvrittr | lsstadtpml | agvseyelpe | dpkwefprdk | |
| 481 | ltlgkplgeg | cfgqvvmaea | vgidkdkpke | avtvavkmlk | ddatekdlsd | lvsememmkm | |
| 541 | igkhkniinl | lgactqdgpl | yviveyaskg | nlreylrarr | ppgmeysydi | nrvpeeqmtf | |
| 601 | kdlvsctyql | argmeylasq | kcihrdlaar | nvlvtennvm | kiadfglard | innidyykkt | |
| 661 | tngrlpvkwm | apealfdrvy | thqsdvwsfg | vlmweiftlg | gspypgipve | elfkllkegh | |
| 721 | rmdkpanctn | elymmmrdcw | havpsqrptf | kqlvedldri | ltlttneeyl | dlsqpleqys | |
| 781 | psypdtrssc | ssgddsvfsp | dpmpyepclp | qyphingsvk | t | | |

Genbank/EMBL /DDBJ Accession No. P11362, from the National Center
for Biotechnology Information - Fibroblast Growth Factor Receptor - 1
(Issacchi A., et al., (1990)) (822 amino acids).

| | | | | | | | |
|---|---|---|---|---|---|---|---|
| 1 | mswkcllfw | avlvtatlct | arpsptlpeq | aqpwgapvev | esflvhpgdl | lqlrcrlrdd | SEQ. ID.: 9 |
| 61 | vqsinwlrdg | vqlaesnrtr | itgeevevqd | svpadsglya | cvtsspsgsd | ttyfsvnvsd | |
| 121 | alpssedddd | dddssseeke | tdntkpnrmp | vapywtspek | mekklhavpa | aktvkfkcps | |
| 181 | sgtpnptlrw | lkngkefkpd | hriggykvry | atwsiimdsv | vpsdkgnytc | iveneygsin | |
| 241 | htyqldvver | sphrpilqag | lpanktvalg | snvefmckvy | sdpqphiqwl | khievngski | |
| 301 | gpdnlpyvqi | lktagvnttd | kemevihirn | vsfedageyt | clagnsigls | hhsawltvle | |
| 361 | aleerpavmt | splyleiiiy | ctgafliscm | vgsvivykmk | sgtkksdfhs | qmavhklaks | |
| 421 | iplrrqvtvs | adssasmnsg | vllvrpsrls | ssgtpmlagv | seyelpedpr | welprdrlvl | |
| 481 | gkplgegcfg | qvvlaeaigl | dkdkpnrvtk | vavkmlksda | tekdlsdlis | ememmkmigk | |
| 541 | hkniinllga | ctqdgplyvi | veyaskgnlr | eylqarrppg | leycynpshn | peeqlsskdl | |
| 601 | vscayqvarg | meylaskkci | hrdlaarnvl | vtednvmkia | dfglardihh | idyykkttng | |
| 661 | rlpvkwmape | alfdriythq | sdvwsfgvll | weiftlggsp | ypgvpveelf | kllkeghrmd | |
| 721 | kpsnctnely | mmmrdcwhav | psqrptfkql | vedldrival | | | |

Materials and Methods

Estrus synchronization and superovulation of donor does used as oocyte donors, and micro-manipulation was performed as described in Gavin W. G. 1996, specifically incorporated herein by reference. Isolation and establishment of primary somatic cells, and transfection and preparation of somatic cells used as karyoplast donors were also performed as previously described supra. Primary somatic cells are differentiated non-germ cells that were obtained from animal tissues transfected with a gene of interest using a standard lipid-based transfection protocol. The transfected cells were tested and were transgene-positive cells that were cultured and prepared as described in Baguisi et al., 1999 for use as donor cells for nuclear transfer. It should also be remembered that the enucleation and reconstruction procedures can be performed with or without staining the oocytes with the DNA staining dye Hoechst 33342 or other fluorescent light sensitive composition for visualizing nucleic acids. Preferably, however the Hoechst 33342 is used at approximately 0.1-5.0 μg/ml for illumination of the genetic material at the metaphase plate.

Goats

The herds of pure- and mixed-breed scrapie-free Alpine, Saanen and Toggenburg dairy goats used for this study were maintained under Good Agricultural Practice (GAP) guidelines.

Isolation of Caprine Fetal Somatic Cell Lines

Primary caprine fetal fibroblast cell lines to be used as karyoplast donors were derived from 35- and 40-day fetuses produced by artificially inseminating 2 non-transgenic female animals with fresh-collected semen from a transgenic male animal. Fetuses were surgically removed and placed in equilibrated phosphate-buffered saline (PBS, Ca⁺⁺/Mg⁺⁺-free). Single cell suspensions were prepared by mincing fetal tissue exposed to 0.025% trypsin, 0.5 mM EDTA at 38° C. for 10 minutes. Cells were washed with fetal cell medium [equilibrated Medium-199 (M199, Gibco) with 10% fetal bovine serum (FBS) supplemented with nucleosides, 0.1 mM 2-mercaptoethanol, 2 mM L-glutamine and 1% penicillin/streptomycin (10,000 I. U. eacb/ml)], and were cultured in 25 cm² flasks. A confluent monolayer of primary fetal cells was harvested by trypsinization after 4 days of incubation and then maintained in culture or cryopreserved.

Sexing and Genotyping of Donor Cell Lines

Genomic DNA was isolated from fetal tissue, and analyzed by polymerase chain reaction (PCR) for the presence of a target signal sequence, as well as, for sequences useful for sexing. The target transgenic sequence was detected by amplification of a 367-bp sequence. Sexing was performed using a zfX/zfY primer pair and Sac I restriction enzyme digest of the amplified fragments.

Preparation of Donor Cells for Embryo Reconstruction

A transgenic female line (CFF6) was used for all nuclear transfer procedures. Fetal somatic cells were seeded in 4-well plates with fetal cell medium and maintained in culture (5% CO₂, 39° C.). After 48 hours, the medium was replaced with fresh low serum (0.5% FBS) fetal cell medium. The culture medium was replaced with low serum fetal cell medium every 48 to 72 hours over the next 7 days. On the 7th day following the first addition of low serum medium, somatic cells (to be used as karyoplast donors) were harvested by trypsinization. The cells were re-suspended in equilibrated M199 with 10% FBS supplemented with 2 mM L-glutamine, 1% penicillin/streptomycin (10,000 I. U. each/ml) 1 to 3 hours prior to fusion to the enucleated oocytes.

Oocyte Collection

Oocyte donor does were synchronized and superovulated as previously described (Gavin W. G., 1996), and were mated to vasectomized males over a 48-hour interval. After collection, oocytes were cultured in equilibrated M199 with 10% FBS supplemented with 2 mM L-glutamine and 1% penicillin/streptomycin (10,000 I.U. each/ml).

Cytoplast Preparation and Enucleation

Oocytes with attached cumulus cells were discarded. Cumulus-free oocytes were divided into two groups: arrested Metaphase-II (one polar body) and Telophase-II protocols (no clearly visible polar body or presence of a partially extruding second polar body). The oocytes in the arrested Metaphase-II protocol were enucleated first. The oocytes allocated to the activated Telophase-II protocols were prepared by culturing for 2 to 4 hours in M199/10% FBS. After this period, all activated oocytes (presence of a partially extruded second polar body) were grouped as culture-induced, calcium-activated Telophase-II oocytes (Telophase-II-Ca) and enucleated. Oocytes that had not activated during the culture period were subsequently incubated 5 minutes in M199, 10% FBS containing 7% ethanol to induce activation and then cultured in M199 with 10% FBS for an additional 3 hours to reach Telophase-II (Telophase-II-EtOH protocol).

All oocytes were treated with cytochalasin-B (Sigma, 5 μg/ml in M199 with 10% FBS) 15 to 30 minutes prior to enucleation. Metaphase-II stage oocytes were enucleated with a 25 to 30 μm glass pipette by aspirating the first polar body and adjacent cytoplasm surrounding the polar body (˜30% of the cytoplasm) to remove the metaphase plate. Telophase-II-Ca and Telophase-II-EtOH oocytes were enucleated by removing the first polar body and the surrounding cytoplasm (10 to 30% of cytoplasm) containing the partially extruding second polar body. After enucleation, all oocytes were immediately reconstructed.

Nuclear Transfer and Reconstruction

Donor cell injection was conducted in the same medium used for oocyte enucleation. One donor cell was placed between the zona pellucida and the ooplasmic membrane using a glass pipet. The cell-oocyte couplets were incubated in M199 for 30 to 60 minutes before electrofusion and activation procedures. Reconstructed oocytes were equilibrated in fusion buffer (300 mM mannitol, 0.05 mM CaCl₂, 0.1 mM MgSO₄, 1 mM K₂HPO₄, 0.1 mM glutathione, 0.1 mg/ml BSA) for 2 minutes. Electrofusion and activation were conducted at room temperature, in a fusion chamber with 2 stainless steel electrodes fashioned into a “fusion slide” (500 μm gap; BTX-Genetronics, San Diego, Calif.) filled with fusion medium.

Fusion was performed using a fusion slide. The fusion slide was placed inside a fusion dish, and the dish was flooded with a sufficient amount of fusion buffer to cover the electrodes of the fusion slide. Couplets were removed from the culture incubator and washed through fusion buffer. Using a stereomicroscope, couplets were placed equidistant between the electrodes, with the karyoplast/cytoplast junction parallel to the electrodes. It should be noted that the voltage range applied to the couplets to promote activation and fusion can be from 1.0 kV/cm to 10.0 kV/cm. Preferably however, the initial single simultaneous fusion and activation electrical pulse has a voltage range of 2.0 to 3.0 kV/cm, most preferably at 2.5 kV/cm, preferably for at least 20 μsec duration. This is applied to the cell couplet using a BTX ECM 2001 Electrocell Manipulator. The duration of the micropulse can vary from 10 to 80 μsec. After the process the treated couplet is typically transferred to a drop of fresh fusion buffer. Fusion treated couplets were washed through equilibrated SOF/FBS, then transferred to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA).

Starting at between 10 to 90 minutes post-fusion, most preferably at 30 minutes post-fusion, the presence of an actual karyoplast/cytoplast fusion is determined. For the purposes of the current invention fused couplets may receive an additional activation treatment (double pulse). This additional pulse can vary in terms of voltage strength from 0.1 to 5.0 kV/cm for a time range from 10 to 80 μsec. Preferably however, the fused couplets would receive an additional single electrical pulse (double pulse) of 0.4 or 2.0 kV/cm for 20 μsec. The delivery of the additional pulse could be initiated at least 15 minutes hour after the first pulse, most preferably however, this additional pulse would start at 30 minutes to 2 hours following the initial fusion and activation treatment to facilitate additional activation. In the other experiments, non-fused couplets were re-fused with a single electrical pulse. The range of voltage and time for this additional pulse could vary from 1.0 kV/cm to 5.0 kV/cm for at least 10 μsec occurring at least 15 minutes following an initial fusion pulse. More preferably however, the additional electrical pulse varied from of 2.2 to 3.2 kV/cm for 20 μsec starting at 30 minutes to 1 hour following the initial fusion and activation treatment to facilitate fusion. All fused and fusion treated couplets were returned to SOF/FBS plus 5 μg/ml cytochalasin-B. The couplets were incubated at least 20 minutes, preferably 30 minutes, at 37-39° C. in a humidified gas chamber containing approximately 5% CO₂ in air.

An additional version of the current method of the invention provides for an additional single electrical pulse (double pulse), preferably of 2.0 kV/cm for the cell couplets, for at least 20 μsec starting at least 15 minutes, preferably 30 minutes to 1 hour, following the initial fusion and activation treatment to facilitate additional activation. The voltage range for this additional activation pulse could be varied from 1.0 to 6.0 kV/cm.

Alternatively, in subsequent efforts the remaining fused couplets received at least three additional single electrical pulses (quad pulse) most preferably at 2.0 kV/cm for 20 μsec, at 15 to 30 minute intervals, starting at least 30 minutes following the initial fusion and activation treatment to facilitate additional activation. However, it should be noted that in this additional protocol the voltage range for this additional activation pulse could be varied from 1.0 to 6.0 kV/cm, the time duration could vary from 10 μsec to 60 μsec, and the initiation could be as short as 15 minutes or as long as 4 hours following initial fusion treatments. In the subsequent experiments, non-fused couplets were re-fused with a single electrical pulse of 2.6 to 3.2 kV/cm for 20 μsec starting at 1 hours following the initial fusion and activation treatment to facilitate fusion. All fused and fusion treated couplets were returned to equilibrated SOF/FBS with or without cytochalasin-B. If cytocholasin-B is used its concentration can vary from 1 to 15 μg/ml, most preferably at 5 μg/ml. The couplets were incubated at 37-39° C. in a humidified gas chamber containing approximately 5% CO₂ in air for at least 30 minutes. Mannitol can be used to substitute for Cytocholasin-B.

Starting at 30 minutes following re-fusion, the success of karyoplast/cytoplast re-fusion was determined. Fusion treated couplets were washed with equilibrated SOF/FBS, then transferred to equilibrated SOF/FBS plus 5 μg/ml cycloheximide. The couplets were incubated at 37-39° C. in a humidified gas chamber containing approximately 5% CO₂ in air for up to 4 hours.

Following cycloheximide treatment, couplets were washed extensively with equilibrated SOF medium supplemented with at least 0.1% bovine serum albumin, preferably at least 0.7%, preferably 0.8%, plus 100 U/ml penicillin and 100 μg/ml streptomycin (SOF/BSA). Couplets were transferred to equilibrated SOF/BSA, and cultured undisturbed for 24-48 hours at 37-39° C. in a humidified modular incubation chamber containing approximately 6% O₂, 5% CO₂, balance Nitrogen. Nuclear transfer embryos with age appropriate development (1-cell up to 8-cell at 24 to 48 hours) were transferred to surrogate synchronized recipients.

Nuclear Transfer Embryo Culture and Transfer to Recipients

All nuclear transfer embryos were co-cultured on monolayers of primary goat oviduct epithelial cells in 50 μl droplets of M199 with 10% FBS overlaid with mineral oil. Embryo cultures were maintained in a humidified 39° C. incubator with 5% CO₂ for 48 hours before transfer of the embryos to recipient does. Recipient embryo transfer was performed as previously described²².

Pregnancy and Perinatal Care

For goats, pregnancy was determined by ultrasonography starting on day 25 after the first day of standing estrus. Does were evaluated weekly until day 75 of gestation, and once a month thereafter to assess fetal viability. For the pregnancy that continued beyond 152 days, parturition was induced with 5 mg of PGF_{2α} (Lutalyse, Upjohn). Parturition occurred within 24 hours after treatment. Kids were removed from the dam immediately after birth, and received heat-treated colostrum within 1 hour after delivery.

Genotyping of Cloned Animals

Shortly after birth, blood samples and ear skin biopsies were obtained from the cloned female animals (e.g., goats) and the surrogate dams for genomic DNA isolation. Each sample was first analyzed by PCR using primers for a specific transgenic target protein, and then subjected to Southern blot analysis using the cDNA for that specific target protein. For each sample, 5 μg of genomic DNA was digested with EcoRI (New England Biolabs, Beverly, Mass.), electrophoreses in 0.7% agarose gels (SeaKem®, ME) and immobilized on nylon membranes (MagnaGraph, MSI, Westboro, Mass.) by capillary transfer following standard procedures known in the art. Membranes were probed with the 1.5 kb Xho I to Sal I hAT cDNA fragment labeled with α-³²P dCTP using the Prime-It® kit (Stratagene, La Jolla, Calif.). Hybridization was executed at 65° C. overnight. The blot was washed with 0.2× SSC, 0.1% SDS and exposed to X-OMA™ AR film for 48 hours.

Milk Protein Analyses

Hormonal induction of lactation for the juvenile female transgenic animals was performed at two months-of-age. The animals were hand-milked once daily to collect milk samples for hAT expression analyses. Western blot and rhAT activity analyses were performed as described (Edmunds, T. et al., 1998).

In the experiments performed during the development of the current invention, following enucleation and reconstruction, the karyoplast/cytoplast couplets were incubated in equilibrated Synthetic Oviductal Fluid medium supplemented with 1% to 15% fetal bovine serum, preferably at 10% FBS, plus 100 U/ml penicillin and 100 μg/ml streptomycin (SOF/FBS). The couplets were incubated at 37-39° C. in a humidified gas chamber containing approximately 5% CO₂ in air at least 30 minutes prior to fusion.

The present invention allows for increased efficiency of transgenic procedures by providing for an additional generation of activated and fused transgenic embryos. These embryos can be implanted in a surrogate animal or can be clonally propagated and stored or utilized. Also by combining nuclear transfer with the ability to modify and select for these cells in vitro, this procedure is more efficient than previous transgenic embryo techniques. According to the present invention, these transgenic cloned embryos can be used to produce CICM cell lines or other embryonic cell lines. Therefore, the present invention eliminates the need to derive and maintain in vitro an undifferentiated cell line that is conducive to genetic engineering techniques.

Thus, in one aspect, the present invention provides a method for cloning a mammal. In general, a mammal can be produced by a nuclear transfer process comprising the following steps:
(i) obtaining desired differentiated mammalian cells to be used as a source of donor nuclei;
(ii) obtaining oocytes from a mammal of the same species as the cells that are the source of donor nuclei;
(iii) enucleating said oocytes;
(iv) transferring the desired differentiated cell or cell nucleus into the enucleated oocyte;
(v) simultaneously fusing and activating the cell couplet to form a transgenic embryo;
(vi) culturing said transgenic embryo until greater than the 2-cell developmental stage; and
(vii) transferring said transgenic embryo into a host mammal such that the embryo develops into a fetus;
wherein said transgenic embryo contains the DNA sequence of a transmembrane receptor protein of interest.

The present invention also includes a method of cloning a genetically engineered or transgenic mammal, by which a desired gene is inserted, removed or modified in the differentiated mammalian cell or cell nucleus prior to insertion of the differentiated mammalian cell or cell nucleus into the enucleated oocyte.

Also provided by the present invention are mammals obtained according to the above method, and offspring of those mammals. The present invention is preferably used for cloning caprines. The present invention further provides for the use of nuclear transfer fetuses and nuclear transfer and chimeric offspring in the area of cell, tissue and organ transplantation.

In another aspect, the present invention provides a method for producing CICM cells. The method comprises:
(i) obtaining desired differentiated mammalian cells to be used as a source of donor nuclei;
(ii) obtaining oocytes from a mammal of the same species as the cells that are the source of donor nuclei;
(iii) enucleating said oocytes;
(iv) transferring the desired differentiated cell or cell nucleus into the enucleated oocyte;
(v) simultaneously fusing and activating the cell couplet to form a transgenic embryo;
(vii) culturing said transgenic embryo until greater than the 2-cell developmental stage; and
(viii) culturing cells obtained from said cultured activated embryo to obtain CICM cells;
wherein said transgenic embryo contains the DNA sequence of a transmembrane receptor protein of interest.

Also CICM cells derived from the methods described herein are advantageously used in the area of cell, tissue and organ transplantation, or in the production of fetuses or offspring, including transgenic fetuses or offspring. Differentiated mammalian cells are those cells, which are past the early embryonic stage. Differentiated cells may be derived from ectoderm, mesoderm or endoderm tissues or cell layers.

An alternative method can also be used, one in which the cell couplet can be exposed to multiple electrical shocks to enhance fusion and activation. In general, the mammal will be produced by a nuclear transfer process comprising the following steps:
(i) obtaining desired differentiated mammalian cells to be used as a source of donor nuclei;
(ii) obtaining oocytes from a mammal of the same species as the cells that are the source of donor nuclei;
(iii) enucleating said oocytes;
(iv) transferring the desired differentiated cell or cell nucleus into the enucleated oocyte;
employing at least two electrical shocks to a cell-couplet to initiate fusion and activation of said cell-couplet into an activated and fused embryo.
(vii) culturing said activated and fused embryo until greater than the 2-cell developmental stage; and
(viii) transferring said first and/or second transgenic embryo into a host mammal such that the embryo develops into a fetus;
wherein the second of said at least two electrical shocks is administered at least 15 minutes after an initial electrical shock.

Mammalian cells, including human cells, may be obtained by well-known methods. Mammalian cells useful in the present invention include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells, i.e., cells useful in the subject invention, may be obtained from any cell or organ of the body. This includes all somatic or germ cells.

Fibroblast cells are an ideal cell type because they can be obtained from developing fetuses and adult animals in large quantities. Fibroblast cells are differentiated somewhat and, thus, were previously considered a poor cell type to use in cloning procedures. Importantly, these cells can be easily propagated in vitro with a rapid doubling time and can be clonally propagated for use in gene targeting procedures. Again the present invention is novel because differentiated cell types are used. The present invention is advantageous because the cells can be easily propagated, genetically modified and selected in vitro.

Suitable mammalian sources for oocytes include goats, sheep, cows, pigs, rabbits, guinea pigs, mice, hamsters, rats, primates, etc. Preferably, the oocytes will be obtained from caprines and ungulates, and most preferably goats. Methods for isolation of oocytes are well known in the art. Essentially, this will comprise isolating oocytes from the ovaries or reproductive tract of a mammal, e.g., a goat. A readily available source of goat oocytes is from hormonal induced female animals.

For the successful use of techniques such as genetic engineering, nuclear transfer and cloning, oocytes may preferably be matured in vivo before these cells may be used as recipient cells for nuclear transfer, and before they can be fertilized by the sperm cell to develop into an embryo. Metaphase II stage oocytes, which have been matured in vivo have been successfully used in nuclear transfer techniques. Essentially, mature metaphase II oocytes are collected surgically from either non-superovulated or superovulated animals several hours past the onset of estrus or past the injection of human chorionic gonadotropin (hCG) or similar hormone.

Moreover, it should be noted that the ability to modify animal genomes through transgenic technology offers new alternatives for the manufacture of recombinant proteins. The production of human recombinant pharmaceuticals in the milk of transgenic farm animals solves many of the problems associated with microbial bioreactors (e.g., lack of post-translational modifications, improper protein folding, high purification costs) or animal cell bioreactors (e.g., high capital costs, expensive culture media, low yields).

The stage of maturation of the oocyte at enucleation and nuclear transfer has been reported to be significant to the success of nuclear transfer methods. (First and Prather 1991). In general, successful mammalian embryo cloning practices use the metaphase II stage oocyte as the recipient oocyte because at this stage it is believed that the oocyte can be or is sufficiently “activated” to treat the introduced nucleus as it does a fertilizing sperm. In domestic animals, and especially goats, the oocyte activation period generally occurs at the time of sperm contact and penetrance into the oocyte plasma membrane.

After a fixed time maturation period, which ranges from about 10 to 40 hours, and preferably about 16-18 hours, the oocytes will be enucleated. Prior to enucleation the oocytes will preferably be removed and placed in EMCARE media containing 1 milligram per milliliter of hyaluronidase prior to removal of cumulus cells. This may be effected by repeated pipetting through very fine bore pipettes or by vortexing briefly. The stripped oocytes are then screened for polar bodies, and the selected metaphase II oocytes, as determined by the presence of polar bodies, are then used for nuclear transfer. Enucleation follows.

Enucleation may be effected by known methods, such as described in U.S. Pat. No. 4,994,384 which is incorporated by reference herein. For example, metaphase II oocytes are either placed in EMCARE media, preferably containing 7.5 micrograms per milliliter cytochalasin B, for immediate enucleation, or may be placed in a suitable medium, for example an embryo culture medium such as CR1aa, plus 10% FBS, and then enucleated later, preferably not more than 24 hours later, and more preferably 16-18 hours later.

Enucleation may be accomplished microsurgically using a micropipette to remove the polar body and the adjacent cytoplasm. The oocytes may then be screened to identify those of which have been successfully enucleated. This screening may be effected by staining the oocytes with 1 microgram per milliliter 33342 Hoechst dye in EMCARE or SOF, and then viewing the oocytes under ultraviolet irradiation for less than 10 seconds. The oocytes that have been successfully enucleated can then be placed in a suitable culture medium.

In the present invention, the recipient oocytes will preferably be enucleated at a time ranging from about 10 hours to about 40 hours after the initiation of in vitro or in vivo maturation, more preferably from about 16 hours to about 24 hours after initiation of in vitro or in vivo maturation, and most preferably about 16-18 hours after initiation of in vitro or in vivo maturation.

A single mammalian cell of the same species as the enucleated oocyte will then be transferred into the perivitelline space of the enucleated oocyte used to produce the activated embryo. The mammalian cell and the enucleated oocyte will be used to produce activated embryos according to methods known in the art. For example, the cells may be fused by electrofusion. Electrofusion is accomplished by providing a pulse of electricity that is sufficient to cause a transient breakdown of the plasma membrane. This breakdown of the plasma membrane is very short because the membrane reforms rapidly. Thus, if two adjacent membranes are induced to breakdown and upon reformation the lipid bilayers intermingle, small channels will open between the two cells. Due to the thermodynamic instability of such a small opening, it enlarges until the two cells become one. Reference is made to U.S. Pat. No. 4,997,384 by Prather et al., (incorporated by reference in its entirety herein) for a further discussion of this process. A variety of electrofusion media can be used including e.g., sucrose, mannitol, sorbitol and phosphate buffered solution. Fusion can also be accomplished using Sendai virus as a fusogenic agent (Ponimaskin et al., 2000).

Also, in some cases (e.g. with small donor nuclei) it may be preferable to inject the nucleus directly into the oocyte rather than using electroporation fusion. Such techniques are disclosed in Collas and Barnes, MOL. REPROD. DEV., 38:264-267 (1994), incorporated by reference in its entirety herein.

The activated embryo may be activated by known methods. Such methods include, e.g., culturing the activated embryo at sub-physiological temperature, in essence by applying a cold, or actually cool temperature shock to the activated embryo. This may be most conveniently done by culturing the activated embryo at room temperature, which is cold relative to the physiological temperature conditions to which embryos are normally exposed.

Alternatively, activation may be achieved by application of known activation agents. For example, penetration of oocytes by sperm during fertilization has been shown to activate perfusion oocytes to yield greater numbers of viable pregnancies and multiple genetically identical calves after nuclear transfer. Also, treatments such as electrical and chemical shock may be used to activate NT embryos after fusion. Suitable oocyte activation methods are the subject of U.S. Pat. No. 5,496,720, to Susko-Parrish et al., herein incorporated by reference in its entirety.

Additionally, activation may best be effected by simultaneously, although protocols for sequential activation do exist. In terms of activation the following cellular events occur:
(i) increasing levels of divalent cations in the oocyte, and
(ii) reducing phosphorylation of cellular proteins in the oocyte.

The above events can be exogenously stimulated to occur by introducing divalent cations into the oocyte cytoplasm, e.g., magnesium, strontium, barium or calcium, e.g., in the form of an ionophore. Other methods of increasing divalent cation levels include the use of electric shock, treatment with ethanol and treatment with caged chelators. Phosphorylation may be reduced by known methods, e.g., by the addition of kinase inhibitors, e.g., serine-threonine kinase inhibitors, such as 6-dimethyl-aminopurine, staurosporine, 2-aminopurine, and sphingosine. Alternatively, phosphorylation of cellular proteins may be inhibited by introduction of a phosphatase into the oocyte, e.g., phosphatase 2A and phosphatase 2B.

Therapeutic Compositions

The proteins of the present invention can be formulated according to known methods to prepare pharmaceutically useful compositions, whereby the inventive molecules, or their functional derivatives, are combined in admixture with a pharmaceutically acceptable carrier vehicle. Suitable vehicles and their formulation, inclusive of other human proteins, e.g., human serum albumin, are described, for example, in order to form a pharmaceutically acceptable composition suitable for effective administration, such compositions will contain an effective amount of one or more of the proteins of the present invention, together with a suitable amount of carrier vehicle.

Pharmaceutical compositions for use in accordance with the present invention may be formulated in conventional manner using one or more physiologically acceptable carriers or excipients. Thus, the recombinant transmembrane receptor proteins and their physiologically acceptable salts and solvate may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they maybe presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the composition may take the form of tablets or lozenges formulated in conventional manner.

For administration by inhalation, the recombinant transmembrane receptor proteins of the invention for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of, e.g. gelatin for use in an inhaler or insufflator may be formulated containing a powder mix of the compound and a suitable powder base such as lactose or starch.

The recombinant transmembrane receptor proteins of the invention may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the recombinant transmembrane receptor proteins of the invention may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Some recombinant transmembrane receptor proteins of the invention may be therapeutically useful in cancer treatment (FGFR 1 through 4). Therefore they may be formulated in conjunction with conventional chemotherapeutic agents or other agents useful in targeting the delivery of the compound of interest. Conventional chemotherapeutic agents include alkylating agents, antimetabolites, various natural products (e.g., vinca alkaloids, epipodophyllotoxins, antibiotics, and amino acid-depleting enzymes), hormones and hormone antagonists. Specific classes of agents include nitrogen mustards, alkyl sulfonates, nitrosoureas, triazenes, folic acid analogues, pyrimidine analogues, purine analogs, platinum complexes, adrenocortical suppressants, adrenocorticosteroids, progestins, estrogens, antiestrogens and androgens. Some exemplary compounds include cyclophosphamide, chlorambucil, methotrexate, fluorouracil, cytarabine, thioguanine, vinblastine, vincristine, doxorubicin, daunorubicin, mitomycin, cisplatin, hydroxyurea, prednisone, hydroxyprogesterone caproate, medroxyprogesterone, megestrol acetate, diethyl stilbestrol, ethinyl estradiol, tamoxifen, testosterone propionate and fluoxymesterone. In treating breast cancer, for example, tamoxifen is preferred.

Accordingly, it is to be understood that the embodiments of the invention herein providing for the transgenic production of transmembrane receptor proteins are merely illustrative of the application of the principles of the invention. It will be evident from the foregoing description that changes in the form, methods of use, and applications of the elements of the disclosed method for the therapeutic use of the claimed transgenic biopharmaceuticals are novel and may be modified and/or resorted to without departing from the spirit of the invention, or the scope of the appended claims.

### Literature Cited and Incorporated by Reference

1. Alberio R, et al., Mammalian Oocyte Activation: Lessons from the Sperm and Implications for Nuclear Transfer, INT J DEV BIOL 2001; 45: 797-809.

2. Alberio R, et al., Remodeling of Donor Nuclei, DNA Synthesis, and Ploidy of Bovine Cumulus Cell Nuclear Transfer Embryos: Effect of Activation Protocol, MOL REPROD DEV2001; 59: 371-379.

3. Baguisi A, et al., Production of Goats by Somatic Cell Nuclear Transfer, NAT BIOTECH 1999; 17: 456-461.

4. Bertoglio D. M., TNF-α Potentiates IL-4/IL-13-induced IL-13R-alpha2 expression, ANN. N. Y. ACAD. Sci. 973: 207-09 (2002).

5. Bondioli K R, Westhusin M E And C R Loony, Production of Identical Bovine Offspring by Nuclear Transfer, THERIOGENOLOGY 1990; 33: 165-174.

6. Brennan, M. B., Drug Discovery. Filtering Out Failures Early In The Pipeline, CHEMICAL & ENGINEERING NEWS, (2000) 5: 63-73.

7. Bronstein, I., et al., Chemiluminescent And Bioluminescent Reporter Gene Assays, ANALYTICAL BIOCHEMISTRY, (1994) 219, 169-81.

8. Campbell, K H S, Mcwhire J, Ritchie W A And I. Wilmut. Sheep Cloned by Nuclear Transfer From a Cultured Cell Line, NATURE 1996; 380: 64-66.

9. Cibelli J B, et al., Cloned Transgenic Calves Produced From Nonquiescent Fetal Fibroblasts. SCIENCE 1998; 280: 1256-1258.

10. Civelli, O., Nothacker, H.-P., & Reinscheid, R., Reverse Physiology:Discovery Of The Novel Neuropeptide, Orphanin FQ/Nociceptin. CRITICAL REVIEWS IN NEUROBIOLOGY, (1998) 12, 163-76.

11. Collas P., Electrically Induced Calcium Elevation, Activation, and Parthenogenic Development of Bovine Oocytes. MOL REPROD 1993; 34: 212-223.

12. Corry D., et al., Induction and Regulation of the IgE Response, NATURE (1999), Supplement to 402 (6760), Pages B18-B23.

13. de Lecea, L. et al., The Hypocretins: Hypothalamus-Specific Peptides With Neuroexcitatory Activity, PROC. NATL. ACAD. SCI. U.S.A. 95 (1), 322-327 (1998).

14. Dionne, C. A., et al., Cloning And Expression Of Two Distinct High-Affinity Receptors Cross-Reacting With Acidic And Basic Fibroblast Growth Factors, EMBO J. 9 (9), 2685-2692 (1990).

15. Drews, J., Drug Discovery: A Historical Perspective, SCIENCE (2000), 287,1960-64.

16. Gavin, W. G., Gene Transfer Into Goat Embryos, TRANSGENIC ANIMALS—GENERATION AND USE, L. M. Houdebine ed., (Harwood Academic Publishers Gmbh., 1996).

17. Grunig G, et al. Requirement for IL-13 independently of IL-4 in experimental asthma SCIENCE 1998 282: 2261-2263.

18. Hill, J., et al., Molecular Cloning And Functional Characterization Of MCH2, A Novel Human MCH Receptor, J. BIOL. CHEM. 276 (23), 20125-29 (2001).

19. Hinuma, S., Onda, H., & Fujino, M., The Quest For Novel Bioactivepeptides Utilizing Orphan Seven-Transmembrane-Domain Receptors, JOURNAL OF MOLECULAR MEDICINE (1999), 77: 495-504.

20. Holgate, S., The Epidemic of Allergy and Asthma, NATURE (1999), Supplement to Volume 402(6760): Pages B2-B4.

21. Holt P. et al., The Role of Allergy in the Development of Asthma, NATURE (1999), Supplement to Volume 402(6760), Pages B12-B17.

22. Isacchi, A., et al., Complete Sequence Of A Human Receptor For Acidic And Basic Fibroblast Growth Factors, NUCLEIC ACIDS RES. 18 (7), 1906 (1990).

23. Kasinathan P, et al., Effect of Fibroblast Donor Cell Age and Cell Cycle on Development of Bovine Nuclear Transfer Embryos In Vitro, BIOL REPROD 2001; 64(5): 1487-1493.

24. Kato Y. et al., Cloning of Calves from Various Somatic Cell Types of Male and Female Adult, Newborn and Fetal Cows, J REPROD FERT 2000; 120: 231-237.

25. Makishima, M., Okamoto, A. Y., Repa, J. J., Tu, H., Learned, R. M., Luk, A., Hull, M. V., Lustig, K. D., Mangelsdorf, D. J., & Shan, B., Identification Of A Nuclear Receptor For Bile Acids, SCIENCE (1999), 284: 1362-65.

26. Marchese, A., George, S. R., Kolakowski, L. F. Jr, Lynch, K. R., & O'Dowd, B. F., Novel GPCRs And Their Endogenous Ligands: Expanding The Boundaries Of Physiology And Pharmacology, TRENDS IN PHARMACOLOGICAL SCIENCES (1999), 20, 370-75.

27. Marshall G D Jr, (moderator); Allergy, Asthma, And Immunology: 60 Years Of Progress, PRESENTED AT: ANNUAL MEETING OF THE AMERICAN ACADEMY OF ALLERGY, ASTHMA, AND IMMUNOLOGY; Mar. 8, 2003; Denver, Colo.

28. Murgue, B., et al., Identification Of A Novel Variant Form Of Fibroblast Growth Factor Receptor 3 (FGFR3 Iiib) In Human Colonic Epithelium, CANCER RES. 54(19), 5206-5211 (1994).

29. Park K W, et al., Developmental Potential of Porcine Nuclear Transfer Embryos Derived from Transgenic Fibroblasts Infected with the Gene for the Green Fluorescent Protein: Comparison of Different Fusion/Activation Conditions, BIOL REPROD 2001; 65: 1681-1685.

30. Partanen, J., et al., FGFR-4, A Novel Acidic Fibroblast Growth Factor Receptor With A Distinct Expression Pattern, EMBO J. 10(6), 1347-54 (1991).

31. Pissios, P., et al., Melanin-Concentrating Hormone Receptor 1 Activates Extracellular Signal-Regulated Kinase And Synergizes With G(S)-Coupled Pathways, ENDOCRINOLOGY 144 (8), 3514-23 (2003).

32. Polejaeva I A, et al., Cloned Pigs Produced by Nuclear Transfer from Adult Somatic Cells, NATURE 2000: 407: 505-509.

33. Sakurai,T., et al., Orexins And Orexin Receptors: A Family Of Hypothalamic Neuropeptides and G Protein-Coupled Receptors That Regulate Feeding Behavior, CELL 92(4), 573-85 (1998).

34. Stice S L, et al., Pluripotent Bovine Embryonic Cell Lines Direct Embryonic Development Following Nuclear Transfer, BIOL REPROD. 1996 Jan; 54(1): 100-10.

35. Wall R J, et al., Transgenic Dairy Cattle: Genetic Engineering on a Large Scale, J DAIRY SCI. 1997 Sep;80(9):2213-24.

36. Willadsen S M, Nuclear Transplantation in Sheep Embryos, NATURE 1986; 320: 63-65.

37. Wilmut I, et al., Viable Offspring Derived From Fetal and Adult Mammalian Cells. NATURE 1997; 385: 810-813.

38. Wu A. H. et al., Molecular cloning and identification of the human interleukin 13 alpha 2 receptor (IL-13Ra2) promoter, NEURO-ONCOLOGY 5(3), 179-187 (2003).

39. Zou X, et al., Production of Cloned Goats from Enucleated Oocytes Injected with Cumulus Cell Nuclei or Fused with Cumulus Cells, CLONING 2001; 3 (1): 31-37.

Patent Applications

St. Croix et a., United States Patent Application 20030017157, ENDOTHELIAL CELL EXPRESSION PATTERNS, filed Jan. 23, 2003.

1. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a transgenic embryo;
(vii) culturing said transgenic embryo(es) until greater than the 2-cell developmental stage; and
(viii) transferring said transgenic embryo into a host mammal such that the embryo develops into a fetus;
wherein the desired differentiated cell or cell nucleus contains a recombinant transgene; and,
wherein said recombinant transgene encodes a recombinant transmembrane receptor protein of interest.

2. The method of claim 1, wherein said donor differentiated mammalian cell to be used as a source of donor nuclei or donor cell nucleus is from mesoderm.

3. The method of claim 1, wherein said donor differentiated mammalian cell to be used as a source of donor nuclei or donor cell nucleus is from endoderm.

4. The method of claim 1, wherein said donor differentiated mammalian cell to be used as a source of donor nuclei or donor cell nucleus is from ectoderm.

5. The method of claim 1, wherein said donor differentiated mammalian cell to be used as a source of donor nuclei or donor cell nucleus is from fetal somatic tissue.

6. The method of claim 1, wherein said donor differentiated mammalian cell to be used as a source of donor nuclei or donor cell nucleus is from fetal somatic cells.

7. The method of claim 1, wherein said donor differentiated mammalian cell to be used as a source of donor nuclei or donor cell nucleus is from a fibroblast.

8. The method of claim 1, wherein said donor differentiated mammalian cell to be used as a source of donor nuclei or donor cell nucleus is from an ungulate.

9. The method of either claims 1 or 8, wherein said donor cell or donor cell nucleus is from an ungulate selected from the group consisting of bovine, ovine, porcine, equine, caprine and buffalo.

10. The method of claim 1, wherein said donor differentiated mammalian cell to be used as a source of donor nuclei or donor cell nucleus is from an adult non-human mammalian somatic cell.

11. The method of claim 1, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

12. The method of claim 1, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

13. The method of claim 1, wherein said at least one oocyte is matured in vivo prior to enucleation.

14. The method of claim 1, wherein said at least one oocyte is matured in vitro prior to enucleation.

15. The method of claim 1, wherein said non-human mammal is a rodent.

16. The method of claim 1, wherein said donor differentiated mammalian cell to be used as a source of donor nuclei or donor cell nucleus is a non-quiescent somatic cell or a nucleus isolated from said non-quiescent somatic cell.

17. The method of either claims 1 or 8, wherein the fetus develops into an offspring.

18. The method of claim 1, wherein said at least one oocyte is enucleated about 10 to 60 hours after initiation of in vitro maturation.

19. The method of claim 1, wherein a desired gene is inserted, removed or modified in said differentiated mammalian cell or cell nucleus prior to insertion of said differentiated mammalian cell or cell nucleus into said enucleated oocyte.

20. The resultant offspring of the methods of claims 1 or 19.

21. The resultant offspring of claim 19 further comprising wherein the offspring created as a result of said nuclear transfer procedure is chimeric.

22. The method of claim 1, wherein cytocholasin-B is used in the cloning protocol.

23. The method of claim 1, wherein cytocholasin-B is not used in the cloning protocol.

24. A method for producing cultured inner cell mass cells, comprising:
(i) obtaining desired differentiated mammalian cells to be used as a source of donor nuclei;
(ii) obtaining at least one oocyte from a mammal of the same species as the cells which are the source of donor nuclei;
(iii) enucleating said at least one oocyte;
(iv) transferring the desired differentiated cell or cell nucleus into the enucleated oocyte;
(v) simultaneously fusing and activating the cell couplet to form a first transgenic embryo;
(vi) activating a cell-couplet that does not fuse to create a first transgenic embryo but that is activated after an initial electrical shock by providing at least one additional activation protocol including an additional electrical shock to form a second transgenic embryo; and
(vi) culturing cells obtained from said cultured activated embryo to obtain cultured inner cell mass cells;
wherein said transgenic embryo encodes a recombinant transmembrane receptor protein of interest.

25. The method of claim 24, wherein said donor differentiated mammalian cell to be used as a source of donor nuclei or donor cell nucleus is from mesoderm.

26. The method of claim 24, wherein said donor differentiated mammalian cell to be used as a source of donor nuclei or donor cell nucleus is from endoderm.

27. The method of claim 24, wherein said donor differentiated mammalian cell to be used as a source of donor nuclei or donor cell nucleus is from ectoderm.

28. The method of claim 24, wherein said donor differentiated mammalian cell to be used as a source of donor nuclei or donor cell nucleus is from fetal somatic tissue.

29. The method of claim 24, wherein said donor differentiated mammalian cell to be used as a source of donor nuclei or donor cell nucleus is from fetal somatic cells.

30. The method of claim 24, wherein said donor differentiated mammalian cell to be used as a source of donor nuclei or donor cell nucleus is from a fibroblast.

31. The method of claim 24, wherein said donor differentiated mammalian cell to be used as a source of donor nuclei or donor cell nucleus is from an ungulate.

32. The method of either claims 24 or 31, wherein said donor cell or donor cell nucleus is from an ungulate selected from the group consisting of bovine, ovine, porcine, equine, caprine and buffalo.

33. The method of claim 24, wherein said donor differentiated mammalian cell to be used as a source of donor nuclei or donor cell nucleus is from an adult mammalian somatic cell.

34. The method of claim 24, wherein said donor differentiated mammalian cell to be used as a source of donor nuclei or donor cell nucleus is selected from the group consisting of epithelial cells, neural cells, epidermal cells, keratinocytes, hematopoietic cells, melanocytes, chondrocytes, B-lymphocytes, T-lymphocytes, erythrocytes, macrophages, monocytes, fibroblasts, and muscle cells.

35. The method of claim 24, wherein said donor differentiated mammalian cell to be used as a source of donor nuclei or donor cell nucleus is from an organ selected from the group consisting of skin, lung, pancreas, liver, stomach, intestine, heart, reproductive organ, bladder, kidney and urethra.

36. The method of claim 24, wherein said at least one oocyte is matured in vivo prior to enucleation.

37. The method of claim 24, wherein said at least one oocyte is matured in vitro prior to enucleation.

38. The method of claim 24, wherein said mammalian cell is derived from a rodent.

39. The method of claim 24, wherein said donor differentiated mammalian cell to be used as a source of donor nuclei or donor cell nucleus is a non-quiescent somatic cell or a nucleus isolated from said non-quiescent somatic cell.

40. The method of either claims 24 or 31, wherein any of said cultured inner cell mass cells fetus develops into a non-human offspring.

41. The method of claim 24, wherein said at least one oocyte is enucleated about 10 to 60 hours after initiation of in vitro maturation.

42. The method of claim 24, wherein a desired gene is inserted, removed or modified in said differentiated mammalian cell or cell nucleus prior to insertion of said differentiated mammalian cell or cell nucleus into said enucleated oocyte.

43. The resultant offspring of the methods of claims 24 or 42.

44. The resultant offspring of claim 42 further comprising wherein any non-human offspring created as a result of said nuclear transfer procedure is chimeric.

45. The method of claim 24, wherein cytocholasin-B is used in the protocol.

46. The method of claim 24, wherein cytocholasin-B is not used in the protocol.

47. The method of claim 24, wherein cytocholasin-B is used in the protocol.

48. The recombinant transmembrane receptor protein of claim 1, wherein said transmembrane protein is the product of a contiguous coding sequence of DNA.

49. The recombinant transmembrane receptor protein of claim 1, wherein said transmembrane protein is expressed in the milk of the host transgenic mammal at a level of at least 1 gram per liter.

50. The recombinant transmembrane receptor protein of claim 1, wherein said transmembrane protein is expressed upon the induction of lactation in mammary epithelial cells.

51. The recombinant transmembrane receptor protein of claim 1, wherein said transmembrane protein, upon expression, retains it biologically activity.

52. The recombinant transmembrane receptor protein of claim 1, wherein said transmembrane protein is engineered to function as a dominant negative version of the native transmembrane protein.

53. The recombinant transmembrane receptor protein of claim 1, wherein said transmembrane protein lacks any biological functionality.

54. The recombinant transmembrane receptor protein of claim 1, wherein said transmembrane protein is selected from the list including: the IL-13 receptor, the Orexin receptor, the melanin concentrating hormone receptor, a fibroblast growth factor receptor, the CFTR receptor, the CD4 receptor and a cadherin.

55. The recombinant transmembrane receptor protein of claim 1, wherein said recombinant transmembrane receptor protein is a dominant negative version of a biological protein selected from the list including: the IL-13 receptor, the Orexin receptor, the melanin concentrating hormone receptor, a fibroblast growth factor receptor, the CFTR receptor, the CD4 receptor and a cadherin.

56. The recombinant transmembrane receptor protein of claim 1, wherein said transmembrane protein is selected from the list including: a channel protein, a drug resistance regulator protein, and an ion pore protein.

57. The recombinant transmembrane receptor protein of claim 24, wherein said transmembrane protein is the product of a contiguous coding sequence of DNA.

58. The recombinant transmembrane receptor protein of claim 24, wherein said transmembrane protein is expressed in the milk of the host transgenic mammal at a level of at least 1 gram per liter.

59. The recombinant transmembrane receptor protein of claim 24, wherein said transmembrane protein is expressed upon the induction of lactation in mammary epithelial cells.

60. The recombinant transmembrane receptor protein of claim 24, wherein said transmembrane protein, upon expression, retains it biologically activity.

61. The recombinant transmembrane receptor protein of claim 24, wherein said transmembrane protein is engineered to function as a dominant negative version of the native transmembrane protein.

62. The recombinant transmembrane receptor protein of claim 24, wherein said transmembrane protein lacks any biological functionality.

63. The recombinant transmembrane receptor protein of claim 24, wherein said transmembrane protein is selected from the list including: the IL-13 receptor, the Orexin receptor, the melanin concentrating hormone receptor, a fibroblast growth factor receptor, the CFTR receptor, the CD4 receptor and a cadherin.

64. The recombinant transmembrane receptor protein of claim 24, wherein said recombinant transmembrane receptor protein is a dominant negative version of a biological protein selected from the list including: the IL-13 receptor, the Orexin receptor, the melanin concentrating hormone receptor, a fibroblast growth factor receptor, the CFTR receptor, the CD4 receptor and a cadherin.

65. The recombinant transmembrane receptor protein of claim 24, wherein said transmembrane protein is selected from the list including: a channel protein, a drug resistance regulator protein, and an ion pore protein.

66. A method for cloning a non-human mammal through a nuclear transfer process comprising:
(i) obtaining desired differentiated mammalian cells to be used as a source of donor nuclei;
(ii) obtaining at least one oocyte from a mammal of the same species as the cells which are the source of donor nuclei;
(iii) enucleating said oocytes;
(iv) transferring the desired differentiated cell or cell nucleus into the enucleated oocyte;
employing at least two electrical shocks to a cell-couplet to initiate fusion and activation of said cell-couplet into an activated and fused embryo.
(vii) culturing said activated and fused embryo until greater than the 2-cell developmental stage;
(viii) transferring said fused embryo into a host mammal such that the embryo develops into a fetus;
wherein the second of said at least two electrical shocks is administered at least 15 minutes after an initial electrical shock;
wherein a desired gene is inserted, removed or modified in said differentiated mammalian cell or cell nucleus prior to insertion of said differentiated mammalian cell or cell nucleus into said enucleated oocyte; and
wherein said desired gene encodes a recombinant transmembrane receptor protein of interest that can be expressed upon induction of lactation in mammary epithelial cells.

67. A method of treating a disease comprising the administering of an effective amount of a transgenically produced transmembrane receptor protein or dominant negative version thereof such that said compound comes into contact with a cell or group of cells which have been or will be exposed to a disease condition where said compound acts to interfere with the continued progression of the disease.

68. The method of claim 67 where said disease is asthma.

69. The method of claim 67 where said disease is an allergy.

70. The method of claim 67 where said disease is psoriasis.

71. The method of claim 67 where said disease is cancer caused by the overproduction of a FGRF.

72. The method of claim 67 where said disease is an inflammation.

73 A method of treating obesity comprising the administering of an effective amount of a transgenically produced transmembrane receptor protein or dominant negative version thereof.

74 The method of claim 67 where said transmembrane receptor protein is selected from the group consisting of: the orexin receptors, the melanin concentrating hormone receptor and the ghrelin receptor.

75. The method of claim 67 wherein the administration of said compounds is accomplished through an oral administration of a pharmaceutical formulation such as a tablet.
